(19) United States
(12) Patent Application Publication
(10) Pub. No.: US 2014/0371723 A1
(43) Pub. Date: Dec. 18, 2014

(54) Rapid Access Vitreal Injection Guide

(71) Applicants: Dennis P. Han, Whitefish Bay, WI (US); Ravi Shankar Singh, Wauwatosa, WI (US)

(72) Inventors: Dennis P. Han, Whitefish Bay, WI (US); Ravi Shankar Singh, Wauwatosa, WI (US)

(21) Appl. No.: 13/918,473

(22) Filed: Jun. 14, 2013

Related U.S. Application Data
(60) Provisional application No. 61/834,589, filed on Jun. 13, 2013.

(51) Int. Cl. A61F 9/00

(52) U.S. Cl. CPC A61F 9/0026
USPC 604/528

(57) ABSTRACT

Embodiments of the invention provide an injection guide for stabilizing an injection site located on an ocular structure during an intravitreal injection. The injection guide includes a first flange positioned opposite a second flange. The first and second flanges can hold portions of the ocular structure away from the injection site. The injection guide can include a handle coupled to the second flange and positioned away from the injection site to provide a substantially unobstructed view of the injection site and the ocular structure. A base plate can be positioned between and coupled to the first and second flange. The base plate includes an aperture or a notch positioned a predetermined distance from an edge of the base plate to provide 360 degree access to the injection site. The base plate can include a tamponade surface configured to tamponade the ocular structure after the intravitreal injection.

### RELATED APPLICATIONS

This application claims priority to U.S. Provisional Application No. 61/834.589 filed on Jun. 13, 2013, the entire disclosure of which is incorporated herein by reference.

### BACKGROUND

Injection of pharmaceutical agents into the vitreous cavity of the eye, commonly known as intravitreal injection, has become a mainstay of treatment of conditions such as macular degeneration, retinal vein occlusions, and diabetic macular edema. Conventional intravitreal injection techniques require the use of several instruments that a user has to manipulate, including an eyelid speculum, measuring calipers, a syringe with needle, and a cotton tipped applicator. The eyelid speculum is used to keep the eyelid margins away from the injection site and to keep the eye open while manual measurements are made to choose a safe injection site that will not harm vital ocular structures. Typically, measuring calipers are coupled to the eyelid speculum to define the extent of the pars plana and identify the site of needle entry in relation to the ocular limbus. The cotton tipped applicator is typically used for post injection tamponade of the injection site. The simultaneous manipulation of the above mentioned instruments adds complexity to the intravitreal injection procedure and can increase the possibility of error and contamination at the injection site.

Several injection assistance devices have been proposed to limit the instruments needed to assist in performing an intravitreal injection; however, these devices have several drawbacks. First, many of the devices still require the use of a separate eyelid speculum which may cause patient discomfort and, with repeated use, could lead to involutional ptosis or ectropion. Some devices also lack a tamponading surface around the injection aperture that allows the user to apply pressure to reduce bleeding or extrusion of the injected agent from the eye. Other injection assistance devices tamponade the injection site by twisting the device over the eye and pushing the eyeball back into the orbit which can be painful and irritating to the ocular surface, even under topical anesthesia. Moreover, such forceful pressure on the eye may distort the normal anatomy of the eyeball and thus cause inadvertent injury to the lens or the retina. Further, many of the proposed injection assistance devices place the user's non-injecting hand close to the injection site, increasing risk of needle stick injury to the user and contamination of the needle. Other devices limit the degrees of freedom in terms of the circumferential location of the injection site around the eye and inhibit visualization of the limbus and ocular structures, requiring the user to be in an awkward head-on over the patient and injection site position in order to visualize and align the injection needle.

### SUMMARY

Some embodiments of the invention provide an injection guide for stabilizing an injection site located on an ocular structure during an intravitreal injection. The injection guide can include a first flange and a second flange that is positioned opposite the first flange. The first and second flanges can hold portions of the ocular structure away from the injection site. The injection guide can include a handle coupled to the second flange and positioned away from the injection site to provide a substantially unobstructed view of the injection site and the ocular structure. A base plate can be positioned between and coupled to the first flange and the second flange. The base plate can have a bottom surface and include an aperture centered on the base plate and positioned a predetermined distance from an edge of the base plate to provide 360 degree access to the injection site. The base plate can include a tamponade surface integral with the bottom surface of the base plate and configured to tamponade the ocular structure after the intravitreal injection.

Other embodiments of the invention provide an injection guide for stabilizing an injection site located on an ocular structure during an intravitreal injection. The injection guide can include a first flange and a second flange positioned opposite the first flange. The first and second flanges can hold portions of the ocular structure away from the injection site. The injection guide can include a handle coupled to the second flange and positioned away from the injection site to provide a substantially unobstructed view of the injection site and the ocular structure. A base plate can be positioned between and coupled to the first flange and the second flange, and the base plate can have a bottom surface. The base plate can include a first notch that extends a predetermined distance from a first edge of the base plate to a central portion of the base plate to provide 360 degree access to the injection site. The base plate can also include a tamponade surface integral with the bottom surface of the base plate and configured to tamponade the ocular structure.

Another embodiment of the invention provides an injection guide for stabilizing an injection site located on an ocular structure during an intravitreal injection with a needle. The injection guide can include a first flange and a second flange positioned opposite the first flange. The first and second flanges can hold portions of the ocular structure away from the injection site. The injection guide can include a handle coupled to the second flange and positioned away from the injection site to provide a substantially unobstructed view of the injection site and the ocular structure. A base plate can be positioned between and coupled to the first flange and the second flange. The base plate can have a bottom surface and include an aperture centered on the base plate and positioned a predetermined distance from an edge of the base plate to provide 360 degree access to the injection site. The base plate can include a needle hub seat axially centered around the aperture and capable of positioning the needle perpendicular to the injection site. A tamponade surface integral with the bottom surface of the base plate can tamponade the ocular structure after the intravitreal injection.

These and other features, aspects, and advantages of the present invention will become better understood upon consideration of the following detailed description, drawings, and appended claims.

### DESCRIPTION OF THE DRAWINGS

FIG. 1 is a top perspective view of an injection guide according to one embodiment of the invention.

FIG. 2 is a side view of the injection guide of FIG. 1 with a handle in-line with a flange of the injection guide.

FIG. 3 is another side view of the injection guide of FIG. 1 with the handle forming an angle with the flange of the injection guide.

FIG. 4 is a perspective view of an injection guide according to another embodiment of the invention.

FIG. 5 is a top view of the injection guide of FIG. 4.

FIG. 6 is a side view of the injection guide of FIG. 4.

FIG. 7 is a top perspective view of an injection guide with a single notch according to another embodiment of the invention.

FIG. 8 is a top perspective view of an injection guide with two notches coincidently aligned according to another embodiment of the invention.

FIG. 9 is a top perspective view of an injection guide with two offset notches according to another embodiment of the invention.

FIGS. 10A and 10B are a top perspective view of an injection guide with a single notch and an aperture according to another embodiment of the invention.

FIG. 11 is a top perspective view of a pediatric injection guide according to another embodiment of the invention.

FIG. 12 is a top perspective view of the injection guide of FIG. 1 with a needle hub seat.

FIG. 13 is a side view of the injection guide of FIG. 12.

FIGS. 14A and 14B are a top perspective view and an end view of an hourglass shaped handle of the injection guide of FIG. 1 according to one embodiment of the invention.

FIGS. 15A and 15B are a top perspective view and an end view of a hexagon shaped handle of the injection guide of FIG. 1 according to one embodiment of the invention.

FIGS. 16A and 16B are a top perspective view and an end view of a tapered handle of the injection guide of FIG. 1 according to one embodiment of the invention.

FIGS. 17A and 17B are a top perspective view and an end view of a thimble coupled to the handle of the injection guide of FIG. 1 according to one embodiment of the invention.

FIG. 18 is a side perspective view of the thimble coupled to the handle of FIG. 17.

FIG. 19 is a side view of an ocular structure receiving a conventional intravitreal injection.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

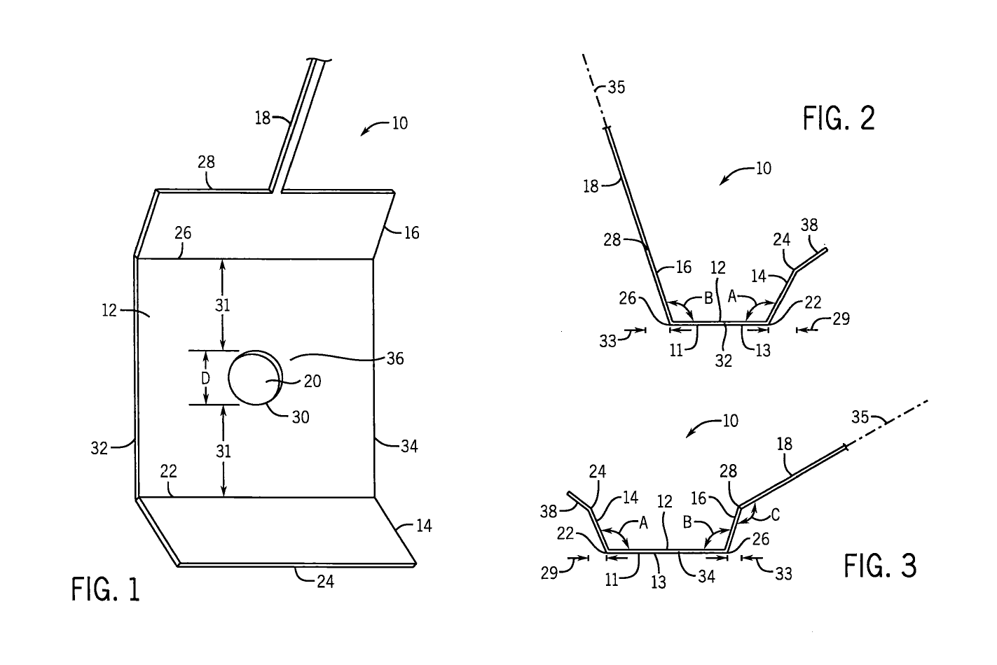

FIG. 1 illustrates an injection guide 10 according to one embodiment of the invention. The injection guide 10 can be a hybrid, single instrument that replaces the speculum, caliper, and cotton tipped applicator used during conventional intravitreal injections. The injection guide 10 can provide rapid, 360 degree access to the injection site at a standard distance from the ocular limbus for treatment of eye diseases using injections of pharmaceuticals and biologics. The injection guide 10 can be reusable or disposable and can be included in a sterilized package (not shown) that includes an intravitreal medication for the intravitreal injection. During operation, a base plate 12 of the injection guide 10 is placed on the bulbar conjunctiva of an ocular structure. Flanges 14, 16, and 38 can hold portions of the ocular adenexa structures, such as eyelids and eyelashes, and keep the injection site clear, thereby obviating the need to pull the eyelids open, as is typically done with a speculum. The injection guide 10 can be used by right or left-handed physicians to inject 360 degrees around the ocular limbus in either the patient's right or left eye.

As shown in FIGS. 1-3, the injection guide 10 can include the base plate 12, the first flange 14, the second flange 16, a handle 18, and an aperture 20. In one embodiment, the base plate 12 is generally flat and square in shape and can have dimensions of about 7 millimeters by about 7 millimeters. The dimensions of the base plate 12 are of substantial size for placement on the bulbar conjunctiva 102 of an ocular structure 100, as shown in FIG. 19. The base plate 12 can have a bottom surface 13 and can further include a tamponade surface 11 integral with the bottom surface 13. The bottom surface 13 of the base plate 12 can be substantially flat or slightly contoured (e.g., slightly concave or slightly convex) to facilitate tamponading. The tamponade surface 11 can be used to tamponade the injection site by gently sliding the tamponade surface 11 over a bulbar conjunctiva 102 after the injection is performed and needle is withdrawn from the eye. The tamponade surface 11 allows the user to apply pressure to reduce bleeding or extrusion of the injected agent from the eye. In an alternative embodiment, the tamponade surface 11 can be textured by, for example, corrugations, striations, or a matte finish to provide relative globe stabilization and simultaneous displacement of the bulbar conjunctiva 102 for injection.

The base plate 12 can be constructed of a thin sheet of metal (e.g., stainless steel) or any suitable type of thermoplastic or polymeric material (e.g., Polyvinyl Chloride (PVC), Polyethylene (PE), low density Polyethylene (LDPE), high density Polyethylene (HDPE), Polypropylene (PP), Polyethylene terephthalate (PET, PETE)). The base plate 12 is of sufficient strength so that the dimensions of the base plate 12 margins remain integral with the device, thereby inhibiting risk of loss of accuracy as can be noted with conventional calipers. The first flange 14 and the second flange 16 can be constructed of similar materials as the base plate 12, and they can be integrally formed with the base plate 12, as will be discussed below.

In one embodiment, the first flange 14 is integrally coupled to the base plate 12 at a first edge 22 of the base plate 12. The first flange 14 can extend outwardly along the entire length of the first edge 22, and can extend upwardly about 2 millimeters or more from the first edge 22 of the base plate 12 to a top edge 24 of the first flange 14. The first flange 14 can form an angle A, as shown in FIGS. 2 and 3, that is greater than about 90 degrees, and preferably between about 100-140 degrees, with the base plate 12. In other words, the top edge 24 of the first flange 14 extends a distance 29 between about 0.5-2 millimeters beyond the first edge 22 of the base plate 12, as shown in FIGS. 2 and 3. keeping the injection site clear of the eyelids and eyelashes during an intravitreal injection.

The second flange 16, similar to the first flange 14, is integrally coupled to the base plate 12 at a second edge 26 of the base plate 12. The second edge 26 of the base plate 12 can be positioned opposite the first edge 22 of the base plate 12. In one embodiment, the second flange 16 can extend outwardly along the entire length of the second edge 26, and can extend upwardly about 2 millimeters or more from the second edge 26 of the base plate 12 to a top edge 28 of the second flange 16. The second flange 16 can form an angle B, as shown in FIGS. 2 and 3, that is greater than about 90 degrees in some embodiments, and between about 100-140 degrees in one embodiment, with the base plate 12. In other words, the top edge 28 of the second flange 16 extends a distance 33 between about 0.5-2 millimeters beyond the second edge 26 of the base plate 12, as shown in FIGS. 2 and 3, further keeping the injection site clear of the eyelids and eyelashes during an intravitreal injection.

As shown in FIGS. 1-3, the handle 18 can be coupled to the top edge 28 of the second flange 16 and positioned away from the injection site to provide an unobstructed view of the injection site and ocular structure 100. The handle 18 can be fastened to the top edge 28 through welding; however, other suitable types of fastening can be used. The handle 18 can be constructed of a suitable metal or polymeric material to provide the user with a sturdy handgrip. The handle 18 inhibits the physician's non-injecting hand away from the path of an injection needle 104, as shown in FIG. 19, and facilitates ergonomic control of the injection guide 10. The handle 18 can be circular about an axis 35, as shown in FIGS. 2 and 3, that extends the length of the handle 18. However, as will be described below, several other ergonomic handles 18 can alternatively be used.

As shown in FIG. 1, the aperture 20 is positioned on a central portion 36 of the base plate 12 and can have a diameter D between about 1-1.5 millimeters for an adult sized injection guide 10. In other words, a predetermined distance 31 from an edge 30 of the aperture 20 to the first edge 22 or second edge 26 of the base plate 12 is about 3-4 millimeters for an adult sized injection guide 10, providing 360 degree access to the injection site. Similarly, the predetermined distance 31 from the edge 30 of the aperture 20 to a third edge 32 or fourth edge 34 of the base plate 12 is also about 3-4 millimeters. Using the dimensions described above, the injection site is positioned about 3-4 millimeters posterior to an ocular limbus 106 (e.g., the standard distance the injection site should be from the ocular limbus for an adult eye) over a region of the pars plana 108, as shown in FIG. 19, by aligning any one of the edges 22, 26, 32, 34 of the base plate 12 with the ocular limbus 106.

Alternatively, the dimensions of the base plate 12 and flanges 14, 16 and the location and diameter of the aperture 20 can be modified to accommodate pediatric intravitreal injections that should account for different ocular dimensions. For pediatric intravitreal injections, the base plate 12 can be about 3 millimeters by about 3 millimeters, and the diameter D of the aperture 20 can be between about 0.5-1.0 millimeters. The predetermined distance 31 from the edge 30 of the aperture 20 to any of the first, second, third, or fourth edges 22, 26, 32, 34 of the base plate 12 can be about 0.5-1.0 millimeters. Using the dimensions described above for the pediatric intravitreal injections, the injection site is advantageously positioned about 0.5-1.0 millimeters posterior to the ocular limbus 106 (e.g., the standard distance the injection site should be from the ocular limbus for a pediatric eye) over the region of the pars plana 108, as shown in FIG. 19, by aligning one of the edges 22, 26, 32, 34 of the base plate 12 with the ocular limbus 106.

During operation, the injection guide 10 is held in the non-injecting hand of the physician. After adequate anesthesia and sterile field preparation, the base plate 12 of the injection guide 10 can be placed on the bulbar conjunctiva 102, as shown in FIG. 19, at the site of the injection, aligning an edge 22, 26, 32, 34 of the base plate 12 with the ocular limbus 106. Either a flanged or non-flanged edge 22, 26, 32, 34 of the base plate 12 can be used for alignment, depending upon whether the injection is in a horizontal or vertical meridian of the eye. Placement of the injection guide 10 is aided by resting the hands on the patient's brow and cheek and using the little finger of both hands to prop the eyelids open. Once the bottom surface 13 of the base plate 12 of the injection guide 10 is placed on the bulbar conjunctiva 102, the physician does not need to pull the eyelids open because the first flange 14 and the second flange 16, positioned on opposing first and second edges 22, 26, keep the base plate 12 and the injection site clear of the eyelids and eyelashes. With the other hand, the physician can inject through the aperture 20 in the base plate 12 so that the injection needle 104 forms about a 90 degree angle with the base plate 12 and the injection site on the pars plana 108. Using the injection guide 10 dimensions as previously discussed, the needle insertion site is positioned about 3-4 millimeters posterior to the ocular limbus 106 over the region of the pars plana 108 of an adult eye. Following the injection the needle 104 is withdrawn and the bottom tamponade surface 11 of the base plate 12 can be gently moved along the ocular surface to tamponade the injection site. The injection guide 10 can then be lifted off the ocular surface.

In another embodiment, as shown in FIGS. 2 and 3, the injection guide 10 can further include the third flange 38 coupled to the top edge 24 of the first flange 14. The third flange 38 can advantageously further inhibit the eyelid and eyelashes from interfering with the injection site. Additionally, as distinguished in FIGS. 2 and 3, the handle 18 can be axially aligned along an axis 35 with the second flange 16, as shown in FIG. 2, or the handle 18 can be misaligned with the second flange 16, as shown in FIG. 3, thereby forming an angle C with the second flange 16. Regardless of the orientation of the handle 18, access to the injection site at a standard distance from the ocular limbus 106 and from 360 degrees is provided.

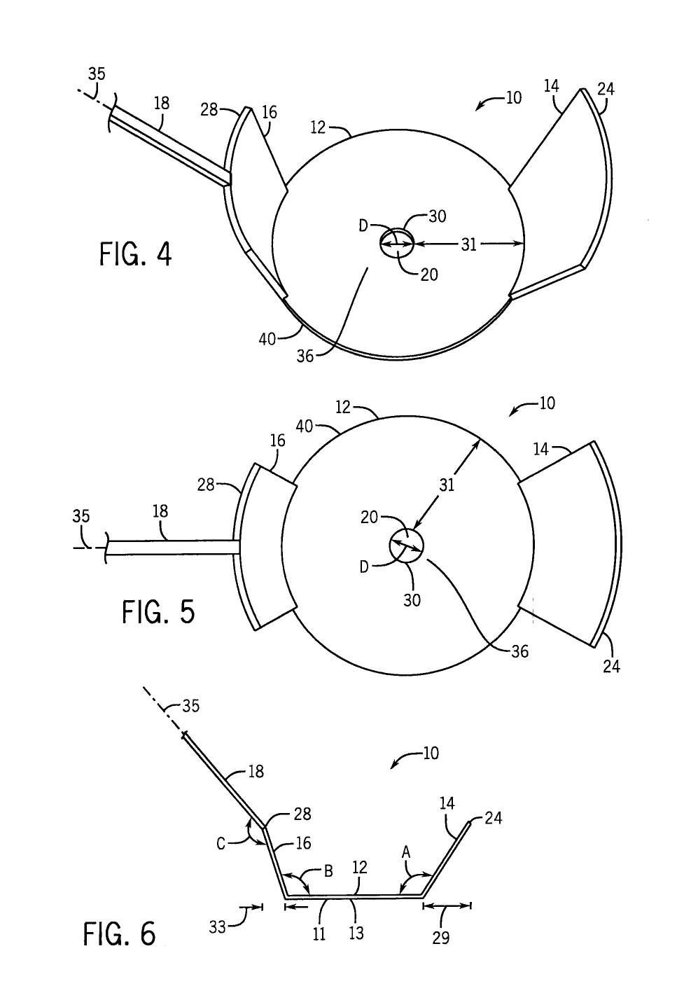

In another embodiment, as shown in FIGS. 4-6, the base plate 12 can alternatively be circular shaped and include the first flange 14 and the second flange 16 positioned opposite one another. The first flange 14 and the second flange 16 can be integrally coupled to a circumferential edge 40 of the base plate 12. The aperture 20 can be positioned in the central portion 36 of the base plate 12 and can have a diameter D between about 1-1.5 millimeters for an adult sized injection guide 10. In other words, as previously described, the predetermined distance 31 from an edge 30 of the aperture 20 to any point on the circumferential edge 40 of the base plate 12 is about 3-4 millimeters for an adult sized injection guide 10. Using the dimensions described above, the injection site is advantageously positioned about 3-4 millimeters posterior to the ocular limbus 106 over the region of the pars plana 108, as shown in FIG. 19, by aligning the circumferential edge 40 of the base plate 12 with the ocular limbus 106.

In an alternative embodiment, the first flange 14 and the second flange 16 can form a continuous flange (not shown) that encompasses the entire circumferential edge 40 of the base plate 12. The continuous flange can keep the injection site clear of the eyelids and eyelashes. If the continuous flange is incorporated into the injection guide 10, the base plate 12 and continuous flange can be made of a transparent, thermoplastic or polymeric material, as discussed above, so that the view of the ocular limbos is not blocked. The other embodiments described can also be constructed of the transparent material to facilitate better visualization of the exterior eye. The predetermined distance 31 from the edge 30 of the aperture 20 to any point on the circumferential edge 40 of the base plate 12 would remain about 3-4 millimeters for an adult sized injection guide 10 having the continuous flange.

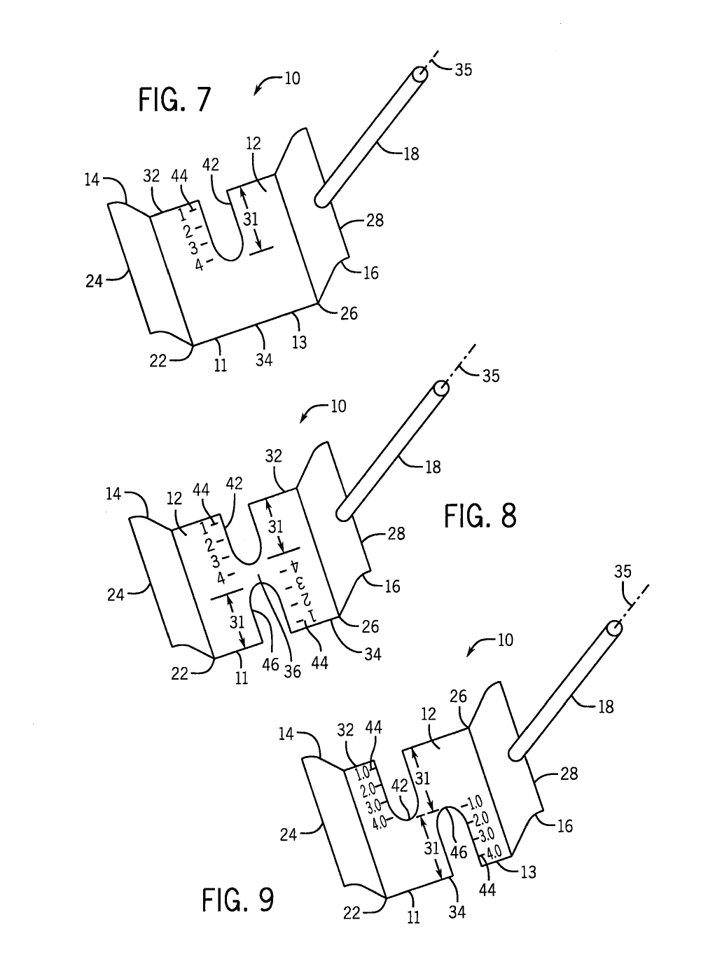

As shown in FIG. 7, the base plate 12 can alternatively have a first notch 42 instead of the aperture 20. The first notch 42 can extend the predetermined distance 31 (e.g., about 3-4 millimeters) from either the third edge 32 or the fourth edge 34 of the base plate 12 to the central portion 36 of the base plate 12. The first notch 42 can include a measuring guide 44 that incorporates “ruler-like” numbers and gradation lines on the base plate 12 of the injection guide 10 for the physicians' reference and to ensure that the injection is performed at the standard distance from the ocular limbus 106, as shown in FIG. 19. With this configuration, the injection guide 10 can be removed from the surgical field after the injection needle 104 is inserted, but before the injection needle 104 is removed, facilitating a two-handed injection. Additionally, the first notch 42 can be positioned to facilitate 360 degree access to the injection site at the standard distance from the ocular limbus 106.

As shown in FIG. 8, the base plate 12 can include both the first notch 42 and a second notch 46 oppositely positioned on the third edge 32 and the fourth edge 34 of the base plate 12. The second notch 46 can extend the predetermined distance 31 (e.g., about 3-4 millimeters) from either the third edge 32 or the fourth edge 34 of the base plate 12 to the central portion 36 of the base plate 12. The second notch 46 can be coincidently aligned with the first notch 42 or, in alternative embodiment, the second notch 46 can be offset from the first notch 42, as shown in FIG. 9. As described above, the second notch 46 can include the measuring guide 44 to ensure that the injection is performed at the standard distance from the ocular limbus 106, as shown in FIG. 19. With this configuration, the base plate 12 of the injection guide 10 may be slightly wider and have dimensions of about 8-9 millimeters by about 6-8 millimeters and would facilitate the physician to approach left or right eyes the same way. Additionally, the first notch 42 and the second notch 46 can be positioned to facilitate 360 degree access to the injection site at the standard distance from the ocular limbus 106.

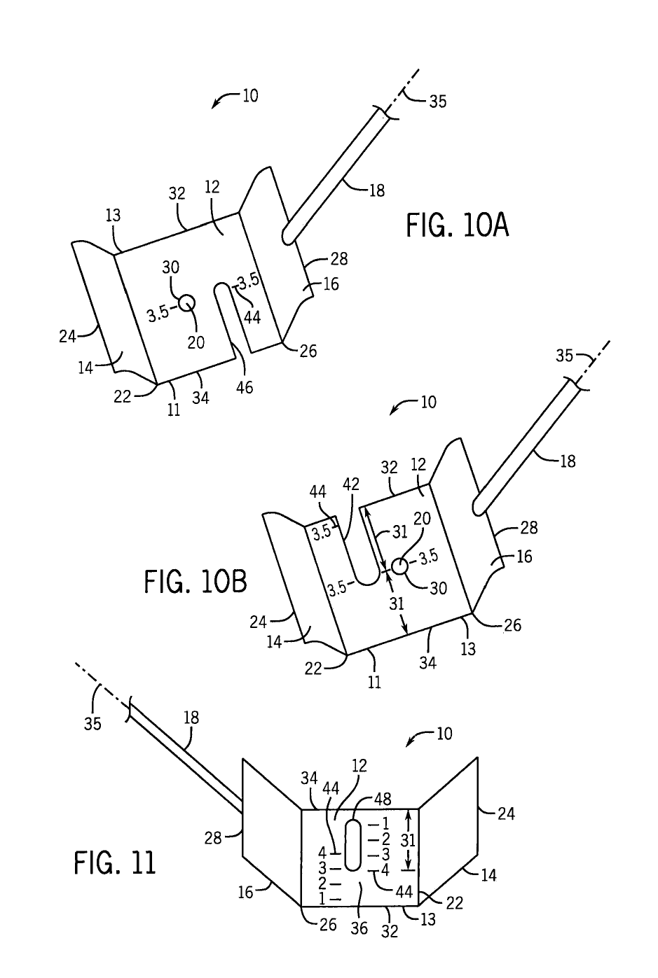

As shown in FIGS. 10A and 10B, an alternative embodiment of the injection guide 10 includes the first notch 42 and the aperture 20 positioned on the base plate 12. As described above, the first notch 42 can extend the predetermined distance 31 (e.g., about 3-4 millimeters) from either the third edge 32 or the fourth edge 34 of the base plate 12 to the central portion 36 of the base plate 12. Similarly, the aperture 20 can be positioned the predetermined distance 31 from the third edge 32 or fourth edge 34 of the base plate 12. The first notch 42 can include a measuring guide 44 that incorporates “ruler-like” numbers and gradation lines on the base plate 12 of the injection guide 10 for the physicians' reference and to ensure that the injection is performed at the standard distance from the ocular limbus 106, as shown in FIG. 19. Additionally, the first notch 42 and the aperture 20 can be positioned to facilitate 360 degree access to the injection site at the standard distance from the ocular limbus 106.

In another alternative embodiment, as shown in FIG. 11, the base plate 12 of the injection guide 10 can be positioned between and coupled to the first flange 14 and the second flange 16. The handle 18 can be coupled to the top edge 28 of the second flange 16 and positioned away from the injection site to provide an unobstructed view of the injection site and ocular structure 100, as shown in FIG. 19. The base plate can include a slit opening 48 eccentrically positioned to facilitate a smaller predetermined distance 31 (e.g., about 1-4 millimeters for pediatric eyes) from either the third edge 32 or the fourth edge 34 of the base plate 12 to the central portion 36 of the base plate 12. The slit opening 48 does not open up to the third edge 32 or the fourth edge 34. Rather, the slit opening 48 is completely enclosed by the base plate 12. As described above, the slit opening 48 can include the measuring guide 44 to ensure that the injection is performed at the standard distance from the ocular limbus 106.

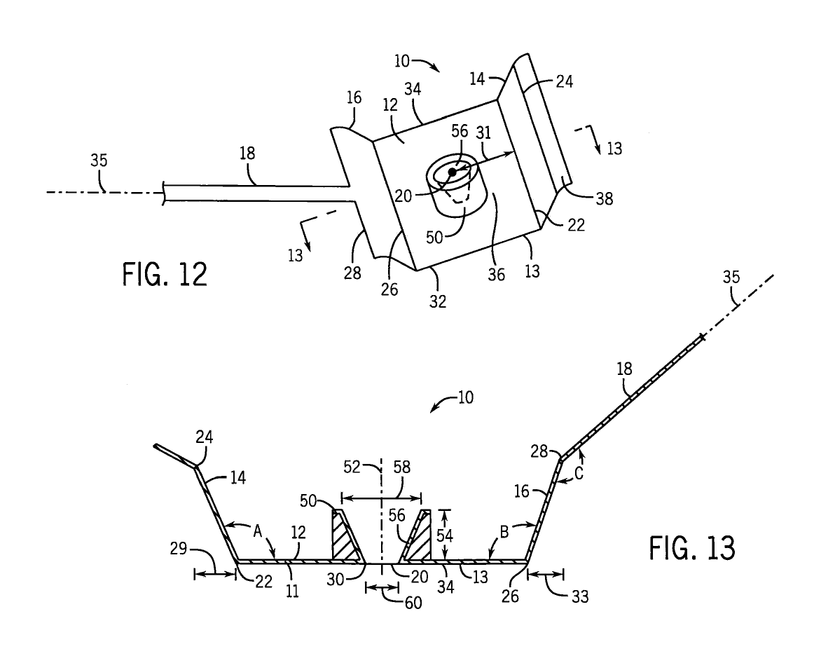

As shown in FIGS. 12 and 13, an alternative embodiment of the injection guide 10 includes a cylindrical shaped needle hub seat 50. Similar to previously discussed embodiments, the base plate 12 of the injection guide 10 can be positioned between and coupled to the first flange 14 and the second flange 16. The handle 18 can be coupled to the top edge 28 of the second flange 16 and positioned away from the injection site. In one embodiment, the needle huh seat 50 can be centered about an axis 52 and encompass the aperture 20 to facilitate positioning the injection needle 104, as shown in FIG. 19, perpendicular to the injection site and also help control needle depth. The needle hub seat 50 can have a height 54 between about 1-2 millimeters and a tapered depression 56, as shown in FIG. 13, with and a top diameter 58 of about 2 millimeters and a bottom diameter 60 of about 1 millimeter. The tapered depression 56 of the needle hub seat 50 can aid in directing the injection needle 104 during an intravitreal injection. The predetermined distance 31 from the edge 30 of the aperture 20 to one of the edges 22, 26, 32, 34 of the base plate 12 is about 3-4 millimeters so that the injection site is positioned 3-4 millimeters posterior to the ocular limbus 106.

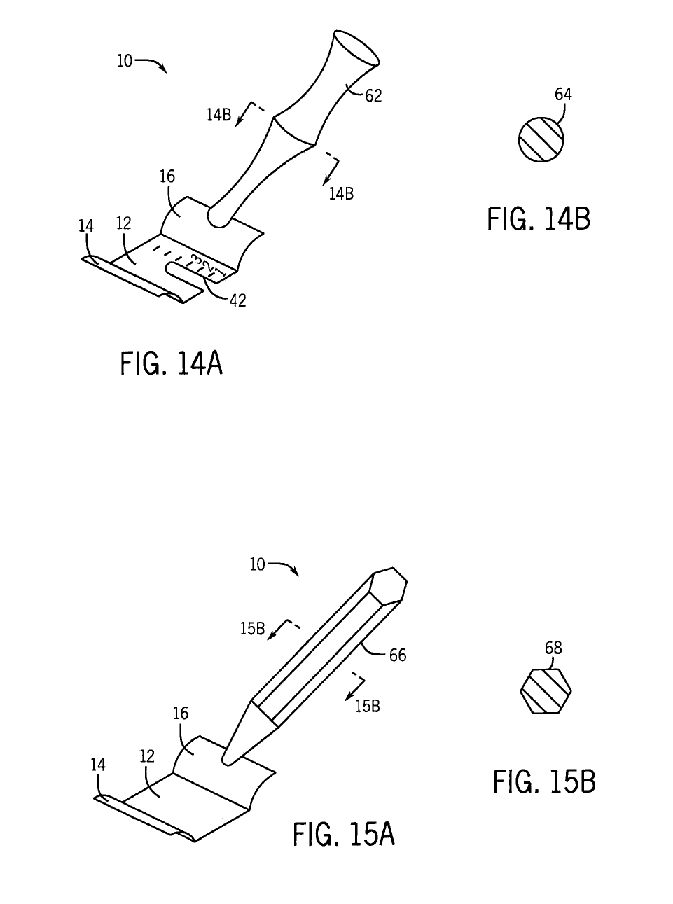

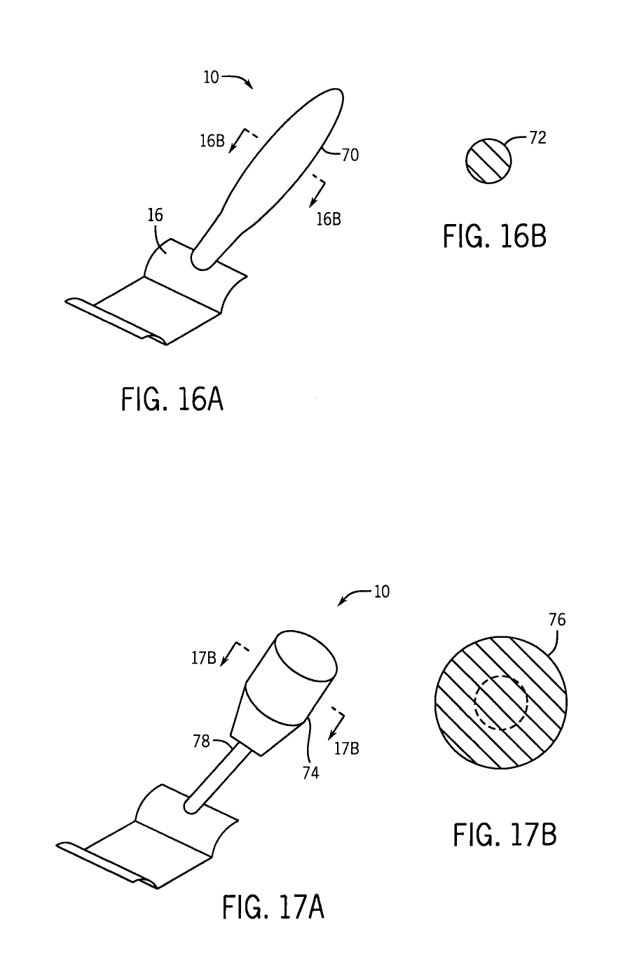

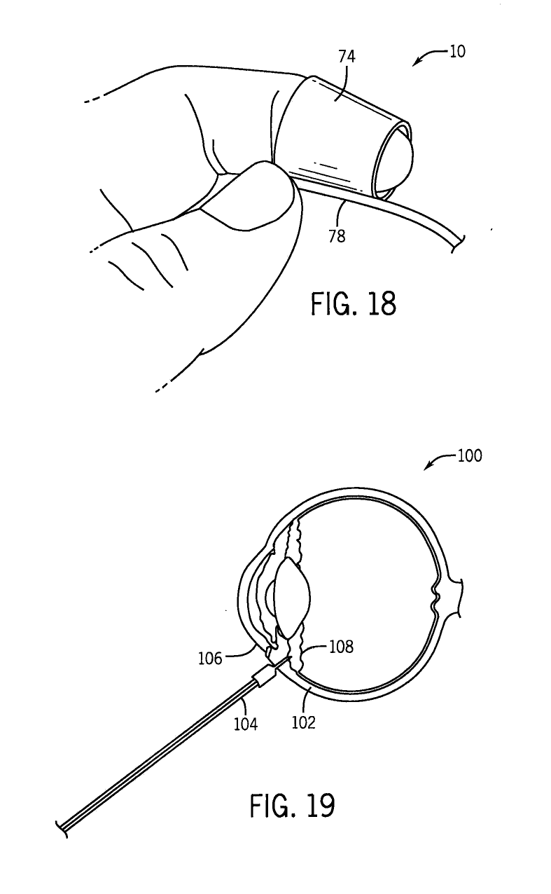

FIGS. 14-18, illustrate several ergonomic handles 18 that can be incorporated into the injection guide 10. For example, as shown in FIG. 14A an hourglass shaped handle 62 can be used to facilitate a stable grip between the thumb and index finger. The hourglass shaped handle 62 can further facilitate a twisting motion, so that the surgeon can spin the injection guide 10 to approach the eye from different directions, yet retain vertical stability. An end view 64 of the hourglass shaped handle 62 is shown in FIG. 14B and is generally circular in shape. Other embodiments can include a hexagon shaped handle 66, as shown in FIG. 15A, that can have an end view 68 that is generally a hexagon shape, as shown in FIG. 15B. Alternatively, as shown in FIG. 16A. a tapered shaped handle 70 can be used having an end view 72 that is generally circular in shape, as shown in FIG. 16B. Or, as shown in FIGS. 17 and 18, a thimble 74 can be coupled to a top portion 78 of the handle 18 and can have an end view 76 that is generally circular in shape.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is Incorporated by reference, as if each such patent or publication were individually incorporated by reference herein. Various features and advantages of the invention are set forth in the following claims.

1. An injection guide for stabilizing an injection site located on an ocular structure during an intravitreal injection, the injection guide comprising:
a first flange;
a second flange positioned opposite the first flange, the first flange and the second flange capable of holding portions of the ocular structure away from the injection site;
a handle coupled to the second flange and positioned away from the injection site to provide a substantially unobstructed view of the injection site and the ocular structure; and
a base plate positioned between and coupled to the first flange and the second flange, the base plate having a bottom surface and including
an aperture centered on the base plate and positioned a predetermined distance from an edge of the base plate to provide 360 degree access to the injection site; and
a tamponade surface integral with the bottom surface of the base plate and configured to tamponade the ocular structure after the intravitreal injection.

2. The injection guide of claim 1 wherein the base plate includes a measuring guide to ensure the intravitreal injection is performed at the predetermined distance from a point on the ocular structure.

3. The injection guide of claim 1 wherein the bottom surface of the base plate is substantially flat.

4. The injection guide of claim 1 wherein the bottom surface of the base plate is slightly contoured.

5. The injection guide of claim 1 wherein a size of the aperture and the predetermined distance are provided for one of an adult injection guide and a pediatric injection guide.

6. The injection guide of claim 5 wherein the predetermined distance is between about 3-4 millimeters for the adult injection guide thereby positioning the injection site about 3-4 millimeters from the point on the ocular structure.

7. The injection guide of claim 1 wherein the injection guide is at least one of a disposable device and a reusable device.

8. The injection guide of claim 1 wherein the injection guide is included in a sterilized package with a intravitreal medication for the intravitreal injection.

9. The injection guide of claim 1 wherein the injection guide is transparent and constructed from at least one of a thermoplastic, polyvinyl chloride (PVC), polyethylene, low density polyethylene (LDPE), high density polyethylene (HDPE), polypropylene (PP), and polyethylene terephalate (PET, PETE).

10. The injection guide of claim 1 wherein the first flange is coupled to the second flange to form a single flange surrounding the entire edge of the base plate.

11. The injection guide of claim 1 wherein the tamponade surface is textured by at least one of corrugations, striations, and a matte finish.

12. The injection guide of claim 1 wherein the handle has one of an hourglass shape, a hexagon shape, and a tapered shape.

13. The injection guide of claim 1 wherein the handle further comprises a thimble coupled to a top portion of the handle.

14. The injection guide of claim 1 and further comprising a third flange coupled to the first flange to further hold the portions of the ocular structure away from the injection site.

15. An injection guide for stabilizing an injection site located on an ocular structure during an intravitreal injection, the injection guide comprising:
a first flange;
a second flange positioned opposite the first flange, the first flange and the second flange capable of holding portions of the ocular structure away from the injection site;
a handle coupled to the second flange and positioned away from the injection site to provide a substantially unobstructed view of the injection site and the ocular structure; and
a base plate positioned between and coupled to the first flange and the second flange, the base plate having a bottom surface and including
a first notch extending a predetermined distance from a first edge of the base plate to a central portion of the base plate to provide 360 degree access to the injection site; and
a tamponade surface integral with the bottom surface of the base plate and configured to tamponade the ocular structure.

16. The injection guide of claim 15 and further comprising a second notch extending the predetermined distance from a second edge of the base plate to the central portion of the base plate, the second notch positioned opposite the first notch.

17. The injection guide of claim 15 wherein the predetermined distance is between about 3-4 millimeters thereby positioning the injection site about 3-4 millimeters from the ocular structure.

18. An injection guide for stabilizing an injection site located on an ocular structure during an intravitreal injection with a needle, the injection guide comprising:
a first flange;
a second flange positioned opposite the first flange, the first flange and the second flange capable of holding portions of the ocular structure away from the injection site;
a handle coupled to the second flange and positioned away from the injection site to provide a substantially unobstructed view of the injection site and the ocular structure; and
a base plate positioned between and coupled to the first flange and the second flange, the base plate having a bottom surface and including
an aperture centered on the base plate and positioned a predetermined distance from an edge of the base plate to provide 360 degree access to the injection site;
a needle hub seat axially centered around the aperture and capable of positioning the needle perpendicular to the injection site; and
a tamponade surface integral with the bottom surface of the base plate and configured to tamponade the ocular structure after the intravitreal injection.

19. The injection guide of claim 18 wherein the needle hub seat includes a tapered depression to inhibit blunting the needle.

20. The injection guide of claim 18 wherein the predetermined distance is between about 3-4 millimeters thereby positioning the injection site about 3-4 millimeters from the ocular structure.
